(19) United States
(12) Patent Application Publication
(10) Pub. No.: US 2026/0083673 A1
(43) Pub. Date: Mar. 26, 2026

(54) OIL-IN-WATER EMULSIONS FOR TOPICAL ADMINISTRATION AND USES THEREOF

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); UNIVERSITE PARIS CITE, Paris (FR); UNIVERSITE PARIS-SACLAY, Gif sur Yvette (FR)

(72) Inventors: Laurence MICHEL, Paris (FR); Maxime SINTÈS, Voisins-le-Bretonneux (FR); Claire ALBERT, Vitry-sur-Seine (FR); Florence AGNELY, Orsay (FR); Mohamed Amine BELADJINE, Colombes (FR); Nicolas HUANG, Orsay (FR); Nicolas TSAPIS, Orsay (FR); Liasmine HAINE HABLAL, Paris (FR)

(21) Appl. No.: 19/113,506

(22) PCT Filed: Sep. 22, 2023
(86) PCT No.: PCT/EP2023/076302

§ 371 Date: Mar. 20, 2025

(30) Foreign Application Priority Data
Sep. 23, 2022 (EP) 22197583.2

(51) Int. Cl. A61K 9/107; A61K 9/51; A61K 31/355; A61K 31/436; A61K 31/593; A61K 31/728; A61K 47/14; A61P 17/06

(52) U.S. Cl. CPC A61K 9/107; A61K 9/5153; A61K 31/355; A61K 31/436; A61K 31/593; A61K 31/728; A61K 47/14; A61P 17/06

(57) ABSTRACT

Oil-in-water Pickering emulsion comprising: an oil phase comprising a first therapeutic agent, an aqueous phase, polyester nanoparticles comprising a second therapeutic agent, wherein the oil phase is in the form of droplets and is dispersed in a continuous aqueous phase, and wherein at least a portion of the nanoparticles are localized at an interface between the oil phase and the aqueous phase, characterized in that the aqueous phase comprises hyaluronan. This new emulsion allows the topical treatment of inflammatory dermatoses such as psoriasis, atopic dermatitis or prurigo, benign skin inflammations such as inflammatory acne, scalp pathologies such asalopecia, dermo-cosmetic conditions, such as very dry irritable skin, tumor pathologies such as mycosis fungoides (indolent cutaneous T lymphoma) or cutaneous mastocytosis (accumulation and abnormal proliferation of mast cells in the dermis, with intense pruritus), and fibrosing pathologies such as keloids (raised, pruritic dystrophic scars, which have the particularity of not regressing spontaneously and of being able to extend beyond the traumatic/injured area).

The present disclosure relates to therapeutic oil-in-water emulsions for topical administration and more particularly to Pickering emulsions co-encapsulating at least two therapeutic agents and their use for the treatment of dermatologic pathologies, such as inflammatory skin diseases.

Topical application of drugs at the pathological site offers the advantage of delivering the drug directly to the site of action and of producing high tissue concentration of the drug. It also allows avoiding unwanted side-effects by other administration routes, like gastric administration in the case of oral delivery of non-steroidal anti-inflammatory drugs.

Emulsions can be used for the topical administration of drugs. They allow the encapsulation of one or several therapeutic agents (in the oil phase and/or the water phase) and offers potential advantages such as the protection of the drugs from degradation, the preservation of their activity, an improved dermal penetration of drugs, or the controlled and sustained release of the drugs. However, due to their thermodynamic instability, emulsions have been widely stabilized by synthetic surfactants, which raises direct or indirect toxicity and environmental issues. In long-term topical treatment, skin irritations are often observed, since surfactants disrupt the skin barrier function.

New stabilization approaches for emulsions have been studied, such as the use of solid particles to stabilize the oil-water interface. These emulsions are called Pickering emulsions and were described more than a century ago by Pickering and Ramsden. They exhibit a long-term stability thanks to solid particles that create a physical barrier against destabilization phenomena, e.g. coalescence.

However, such emulsions have the tendency of creaming over time, i.e. the dispersed phase tends to migrate to the surface of the emulsion, thereby creating inhomogeneity within the emulsion.

The present disclosure provides a new therapeutic oil-in-water emulsion that is stable over time, particularly for at least 24 hours.

The present disclosure provides a new stable oil-in-water emulsion possessing immunomodulatory and anti-inflammatory properties.

The present disclosure provides a new stable oil-in-water emulsion comprising at least one anti-inflammatory agent and having a multipurpose effectiveness superior to that of conventional treatments.

The present disclosure provides a new oil-in-water emulsion that can be loaded with different therapeutic agents, that can be used in topical application and that potentially has cosmetic virtues.

The present disclosure provides a new oil-in-water emulsion that is biodegradable, biocompatible and potentially offers better compliance for users due to the integration of several active agents in a single emulsion.

The present disclosure provides a new oil-in-water emulsion which has a very good texture, i.e. not too liquid or too thick, which makes the application of the treatment pleasant for the patients, and which texture is reproducible.

The present disclosure provides a new oil-in-water emulsion which penetrates the skin correctly for the active ingredients to be effective.

The present disclosure provides a new oil-in-water emulsion which ensures good hydration of the skin after application, which is an essential condition for a good treatment of inflammatory dermatoses, such psoriasis, especially for mature skin.

Thus, the present disclosure relates to an oil-in-water (O/W) Pickering emulsion comprising:
an oil phase comprising a first therapeutic agent,
an aqueous phase,
polyester nanoparticles comprising a second therapeutic agent,
wherein the oil phase is in the form of droplets and is dispersed in a continuous aqueous phase, and wherein at least a portion of the nanoparticles are localized at an interface between the oil phase and the aqueous phase,
characterized in that the aqueous phase comprises hyaluronan.

Hyaluronan refers herein to all physiological forms of hyaluronic acid, the most common of which is the sodium salt.

In the herein disclosed emulsion the pH of the emulsion may be of 4.5 to 6.5, in particular of 5 to 6.

The pH of the emulsion is measured by simply plunging the pH electrode in the emulsion.

In this range, hyaluronan is predominantly in the ionized form (e.g. as sodium hyaluronate) and therefore provides its thickening effect the most efficiently. Moreover, this range of pH is particularly adapted to a topical application of the emulsion on the skin since the natural pH of the skin is around 5.5.

In the herein disclosed emulsion, one role of the nanoparticles is to stabilize the emulsion by preventing or slowing down coalescence of the oil droplets and/or Ostwald ripening. In the herein disclosed emulsion, the nanoparticles make it possible to avoid using surfactants to stabilize the emulsion. Thus, the herein disclosed emulsion may not comprise surfactants, which is advantageous for pharmaceutical and cosmetic applications.

Such an emulsion, stabilized by nanoparticles located at the interface between the oil phase and the water phase is called a Pickering emulsion.

Hydrophilic nanoparticles favor the formulation of O/W Pickering emulsions (rather than W/O emulsion). Moreover, if there is an excess of nanoparticles not adsorbed on the interface between the oil phase and the water phase, this excess will be found mainly in the water phase.

In the herein disclosed emulsion, hyaluronan acts as a thickener that stabilizes the emulsion by slowing down creaming of the emulsion and/or coalescence and ripening processes. Although adding a thickener in an emulsion to stabilize it is known in the art, it remains difficult to predict how the thickener will influence the interfacial properties of the emulsion, especially when the emulsion is a Pickering emulsion. For example, if the thickener tends to adsorb on the particles or at the oil-water interface it could destabilize the emulsion.

Hyaluronan also has moisturizing, soothing and anti-aging properties.

Here it was found that hyaluronan was efficient in stabilizing the emulsion and did not have adverse effects on the emulsion properties. Moreover, in vivo studies show that the herein disclosed emulsion stabilized with hyaluronan have a high potential in treating skin diseases.

In the herein disclosed emulsion, the oil phase may comprise at least one oil chosen from fatty acids, fatty acid esters, vegetable oils, mineral oils, and mixtures thereof.

As defined herein, an oil is a non-aqueous nonpolar chemical substance that composed primarily of hydrocarbons. It is both hydrophobic (does not mix well with water) and lipophilic (mixes well with other oils). It is liquid at 25° C. and 1.013 bar.

In the herein disclosed emulsion, the vegetable oils may be selected from castor oil, sesame oil, poppyseed oil, soybean oil, olive oil, walnut oil, coconut oil, and mixtures thereof.

Other vegetable oils suitable for use in the herein disclosed emulsion comprise safflower oil and palm kernel oil.

Fatty acid esters suitable for use in the herein disclosed emulsion comprise triglycerides, in particular triglycerides of long-chain fatty acids (TLCFA) and/or triglycerides of medium-chain fatty acids (TMCFA).

One example of a TLCFA suitable for use in the herein disclosed emulsion is triolein.

Examples of TMCFA suitable for use in the herein disclosed emulsion include triglycerides of caprylic and/or capric acid. TMCFA suitable for use in the herein disclosed emulsion include MIGLYOL 810® or 812 N®, NEOBEER M-5, Captex® 300, and Labrafac® lipophile WL 1349.

Other triglycerides suitable for use in the herein disclosed emulsion comprise triglycerides of caprylic, capric and linoleic acid (MIGLYOL 818®) and triglycerides of caprylic, capric and succinic acid (MIGLYOL 829®).

Other fatty acid esters suitable for use in the herein disclosed emulsion comprise propylene glycol dicaprylate/dicaprate (MIGLYOL 840®), propylene glycol dicaprylate/dicaprate (Labrafac® PG), and glyceryl oleates (Peceol®).

In the herein disclosed emulsion, the fatty acid esters may be selected from triglycerides of medium-chain fatty acids, in particular the medium-chain fatty acids may comprise between 8 and 12 carbon atoms.

The herein disclosed emulsion may comprise from 5% to 70% by mass of the oil phase, relative to the total mass of the emulsion.

In the herein disclosed emulsion, the droplets forming the oil phase may have a size of at least 1 μm.

In the herein disclosed emulsion, the droplets forming the oil phase may have a size distribution ranging from 1 μm to 100 μm, as measured by dynamic light scattering.

Polyester nanoparticles have a low toxicity and induce only a limited inflammatory response.

Moreover, in the herein disclosed emulsion, the polyester nanoparticles may be biodegradable.

Thus, the herein disclosed emulsion may be biocompatible, biodegradable and less toxic or irritating than emulsions stabilized with surfactants or mineral particles.

In the herein disclosed emulsion, the polyester nanoparticles may be solid nanoparticles (i.e. without an internal cavity). In particular, they may have at least two dimensions of less than 1 μm. In particular, the polyester nanoparticles may be spherical solid nanoparticles with a diameter of less than 1 μm, in particular of 20 nm to 400 nm, as measured by dynamic light scattering.

In this case, the diameter is a z-average, i.e. the intensity weighted mean hydrodynamic size of the ensemble collection of particles measured by dynamic light scattering (DLS).

The concentration of the nanoparticles in the herein disclosed emulsion may be of 5 mg/mL to 40 mg/mL of emulsion.

In the herein disclosed emulsion, the polyester may be selected from polylactic acid, polyglycolic acid, copolymers of lactic acid and glycolic acid, copolymers of lactic acid, glycolic acid and ethylene glycol, polyorthoesters, polyanhydrides, polylactones, such as polybutyrolactone or polyvalerolactone, polymalic acid, and mixtures thereof.

In particular, when the polyester is poly(lactic-co-glycolic acid), poly(lactic-co-glycolic acid) may have a ratio lactic acid/glycolic acid of 1 to 6, preferably of 2 to 4.

In the herein disclosed emulsion, the polyester may be a poly(lactic-co-glycolic acid) having a ratio lactic acid/glycolic acid of 2 to 4 and an intrinsic viscosity of 0.25 dL/g to 0.5 dL/g, as measured at 25° C. at a concentration of 0.1% by mass in CHCl₃.

In the herein disclosed emulsion, the first therapeutic agent may be selected from anti-inflammatory agents such as calcitriol, calcipotriol, maxacalcitol, curcumin, glucocorticoids, in particular betamethasone or dexamethasone, and natural of semisynthetic analogs derived from metabolites of vitamin E.

In the herein disclosed emulsion, the second therapeutic agent may be selected from immunosuppressive agents, such as calcineurin inhibitors, in particular tacrolimus and cyclosporin A, or such as JAK/STATinhibitors, in particular Tofacitinib, for example Tofacitinib citrate, baricitinib, for example baricitinib phosphate salt, and Ruxolitinib, or such as steroidal inhibitors, in particular Cucurbitacin B hydrate, or such as STAT inhibitors, in particular and Stattic (CAS Number: 19983-44-9).

In the herein disclosed emulsion, the oil phase may further comprise an antioxidant such as vitamin E, vitamin C, resveratrol, or N-acetylcystein, in particular vitamin E.

In the herein disclosed emulsion, the hyaluronan may have a molar mass of 5000 g/mol to 5000000 g/mol, in particular of 100000 g/mol to 3000000 g/mol, more particularly of 500000 g/mol to 2500000 g/mol, even more particularly of 1000000 g/mol to 2500000 g/mol.

In the herein disclosed emulsion, the concentration of hyaluronan in the aqueous phase calculated as the mass of hyaluronan in grams contained in 100 mL of the aqueous phase may be of 0.15% to 5%, for example of 0.2% to 2% or of 0.5% to 5%, in particular of 0.25% to 1.5% or of 1% to 3%.

The above concentrations of hyaluronan, allow to obtain stable emulsions with satisfactory viscosities.

The herein disclosed emulsion may comprise Tacrolimus as the first therapeutic agent and calcitriol as he second therapeutic agent. Particularly good results were obtained with this embodiment of the herein disclosed emulsion. Specifically, and surprisingly, in this case, in vivo studies have shown that the herein disclosed emulsion is more efficient than a comparable emulsion wherein hyaluronan has been replaced with Carbopol as a thickening agent.

Carbopol, also called carbomer, is a synthetic biocompatible polymer having a high molar mass, comprising repeating acrylic acid units. In Carbopol, the polymeric chains are cross-linked with allylic saccharose or other allylic esters. Generally, more than 50% of the monomeric units in Carbopol comprise a carboxylic acid group.

The advantages of encapsulating the second therapeutic agent in the nanoparticle include improved skin penetration, creation of a therapeutic agent reservoir in the hair follicles, protection of the encapsulated therapeutic agent and gradual release of the therapeutic agent.

The advantages of the oil phase are the protection and gradual release of the therapeutic agent: indeed, encapsulation in the oil phase is advantageous in that it allows the protection and thus the stabilization of certain types of therapeutic agents, especially fragile molecules. The presence of nanoparticles at the interface between the external aqueous phase and the internal oil phase reinforces the stability of the emulsions and the protection of the therapeutic agent.

The advantages of the combination of the two therapeutic agents and hyaluronan distributed in the three phases of the emulsion (aqueous, oil and nanoparticles) include a progressive and varied release kinetics of the therapeutic agents and of hyaluronan, providing superior efficacy to each of the therapeutic agents and hyaluronan used separately, with better compliance of the combined treatment compared to three separate applications.

The present disclosure, according to another one of its aspects, also relates to a pharmaceutical composition comprising the herein disclosed emulsion and at least one pharmaceutically acceptable excipient.

These pharmaceutical compositions contain an effective dose of at least one emulsion as disclosed herein, as well as at least one pharmaceutically acceptable excipient.

The excipients are selected according to the pharmaceutical form and the desired mode of administration, from the usual excipients known to the person skilled in the art.

The mode of administration can be topical application, subcutaneous injection, or intradermal injection into an area of injured skin.

In accordance with usual practice, the appropriate dosage for each patient is determined by the physician based on the mode of administration, the weight and the response of the patient.

The present disclosure, according to another one of its aspects, also relates to the herein disclosed emulsion for use in a method for treatment of the human or animal body, in particular the treatment of dermatologic pathologies

The present disclosure, according to another one of its aspects, also relates to a treatment method for treating dermatologic pathologies comprising the administration to a patient of an effective dose of the herein disclosed emulsion.

According to the present disclosure, the dermatologic pathologies may comprise:
inflammatory dermatoses such as psoriasis, atopic dermatitis or prurigo,
benign skin inflammations such as inflammatory acne,
dermo-cosmetic conditions, such as very dry irritable, pruriginous skin, scalp pathologies such as various hair loss disorders including alopecia aerate, lichen planus, . . .
cutaneous mastocytosis (accumulation and abnormal proliferation of mast cells in the dermis, with intense pruritus),
fibrosing pathologies such as keloids (raised, pruritic dystrophic scars, which have the particularity of not regressing spontaneously and of being able to extend beyond the traumatic/injured area).
tumor pathologies such as mycosis fungoides (indolent cutaneous T lymphoma) or basal carcinoma.

### EXAMPLES

### Chemicals

Poly(lactide-co-glycolide) acid (PLGA, 75:25 Resomer@ RG753 H with acid ending groups (inherent viscosity: 0.32-0.44 dL/g) was purchased from Evonik (Germany).
Miglyol 812 N was purchased from Cremer Oleo GmbH & Co (Germany).
Tacrolimus monohydrate (TAC) and cyclosporine A (CysA) were purchased from INRESA (France). Calcitriol (CAL) was purchased from Bertin Pharma (France).
α-Tocopherol (Vit E) (purity≥96%), calcein and phosphoric acid were purchased from Sigma-Aldrich (France).
Carbopol 974P was purchased from Lubrizol (France).
Hyaluronan (molar mass 1 550 000 g/mol) was purchased from ACROS organics.

### Preparation of Nanoparticles (NP)

PLGA nanoparticles were prepared by the emulsion-evaporation method or by the nanoprecipitation method (see C. E. Astete, C. M. Sabliov, Synthesis and characterization of PLGA nanoparticles, J. Biomater. Sci. Polym. Ed. 17 (2006) 247-289 and C. Albert, N. Huang, N. Tsapis, S. Geiger, V. Rosilio, G. Mekhloufi, D. Chapron, B. Robin, M. Beladjine, V. Nicolas, E. Fattal, F. Agnely, Bare and sterically stabilized PLGA nanoparticles for the stabilization of Pickering emulsions, Langmuir. 34 (2018) 13935-13945).

In the emulsion-evaporation process, PLGA was dissolved in a dichloromethane/acetone mixture and emulsified by sonication with an aqueous solution of PVA (polyvinyl alcohol). After evaporation of the organic solvent at room temperature, the NPs were purified by ultracentrifugation. After removal of the supernatant, the nanoparticles were resuspended in an aqueous solution containing trehalose (cryoprotector). Then, the NP suspension was lyophilized. Before use, the lyophilized NPs were redispersed in MilliQ water to the desired concentration.

In the nanoprecipitation process, an organic solution of PLGA dissolved in acetonitrile is injected into a PVA solution using a syringe pump. The evaporation of the organic solvent is done at room temperature under the hood. Subsequently, the purification of the nanoparticles was done by ultracentrifugation. After the purification step, and NPs were redispersed in MilliQ water to the desired concentration.

PLGA nanoparticles with SA were prepared by adding the second therapeutic agent (for example, cyclosporin A (CysA) or tacrolimus (TAC) or Tofacitinib citrate (Tofa) or a phosphate salt of Baricitinib (Bari) or Ruxolitinib (Ruxo)) at 10% by weight of the polymer to the PLGA/organic solvent mixture.

The above process gave encapsulation efficiencies (mass of active substance in the NP/total mass of active substance used) above 75% and loading charges (mass of active substance in the NP/total mass of the NP) allowing a biological effect to be obtained (said loading charge depends on the active substance).

Control NP were prepared without active pharmaceutical ingredients (API) following the same method.

### Preparation of a Stabilized Water-in-oil Emulsion at a Concentration of 25 Mg/ml of PLGA Nanoparticles With an Oil Phase/water Phase Ratio of 20/80 by Mass

3 μg and 10 μg per g of emulsion of CAL and vitamin E respectively were added to the oil phase. To each NP suspension (PLGA, PLGA-CysA and PLGA TAC), a more concentrated solution of Carbopol or hyaluronan was added to obtain the aqueous phase with a concentration of 0.2% w/vol in Carbopol (i.e. 0.2 g of Carbopol per 100 ml of aqueous phase) or 1.5% w/vol in hyaluronan in the aqueous suspension (i.e. 1.5 g of HA per 100 mL of the aqueous phase). The aqueous phase and the oil are then mixed to obtain the emulsions, using an Ultra-Turrax (IKA T10) at 20,000 rpm for 2 min. For the emulsion with Carbopol, 15 seconds before the end of the Ultra-Turrax emulsification, three drops of 0.25 M sodium hydroxide (NaOH) solution were added to the emulsions using a Pasteur pipette to neutralize the thickener and induce its gelation.

### Stability of Emulsions With or Without Hyaluronan (ha) in the External Phase

The emulsions were formulated with a 25 mg/mL aqueous suspension of PLGA nanoparticles and with a ratio oil phase (Miglyol)/water phase of 20/80 by mass, with or without active substances (TAC or Cys A in the nanoparticles and CAL in the oil phase, or no active substance in the emulsion). Emulsions without any active substance are called white emulsions. The influence of the addition of HA with a molar mass of 1.5 MDa in the external phase of the emulsion was studied.

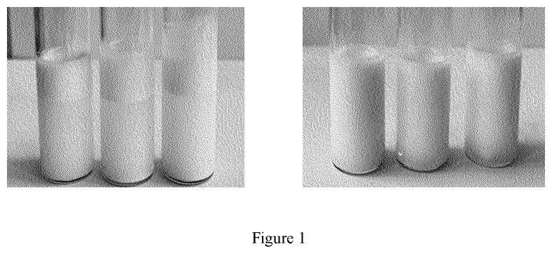

Photographs of the emulsions were taken to visually appreciate their behavior (FIG. 1). The emulsions were also analyzed using a Turbiscan® MA 2000 (Formulaction, Toulouse, France). Measurements were made at determined times by recording the backscattered and/or transmitted light intensity curves. The Turbiscan allows to measure the destabilization phenomena in the sample without dilution. The rheological behavior of the emulsions was measured with an AR-G2 rheometer (TA instruments, USA) equipped with a flat plane geometry (diameter 40 mm, air gap 100 μm). The temperature was controlled with a Peltier plane at 20° C.

FIG. 1 shows the emulsions on day 1. Creaming was visually observed as early as day 1 with the emulsions without HA (left), while the presence of HA at 1.5% by mass in the external phase results in an emulsion layer over almost the entire sample height (right).

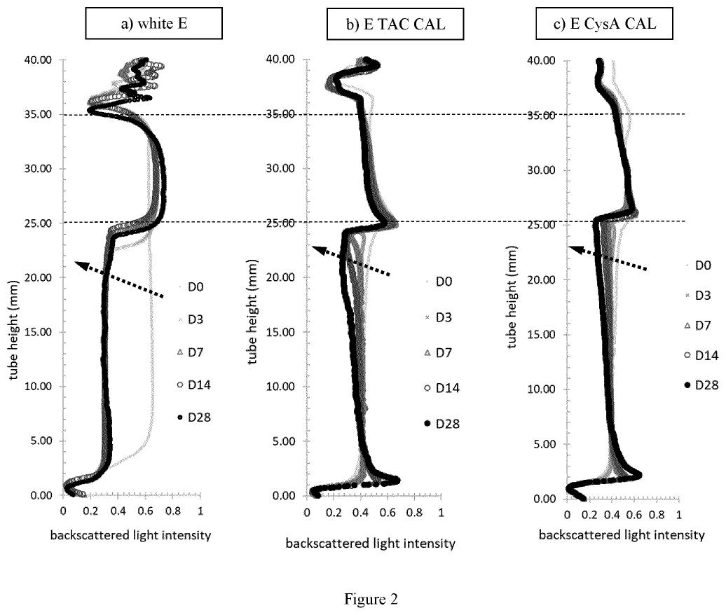

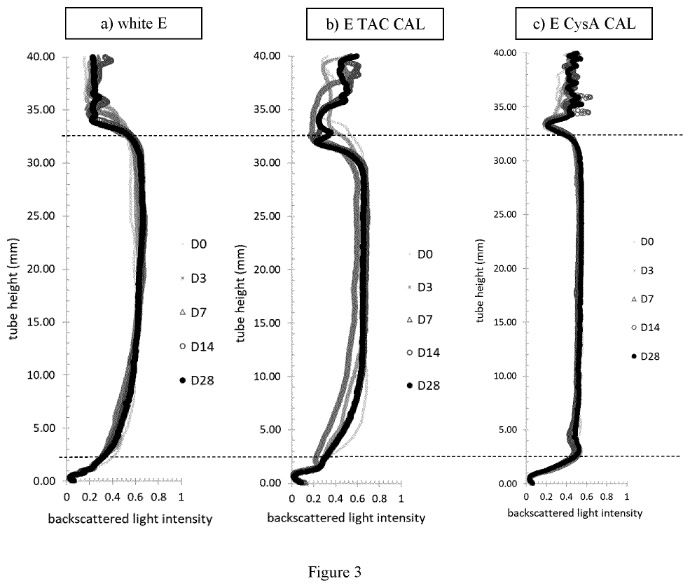

The stability of the emulsions without and with HA was monitored using a Turbiscan (FIGS. 2 and 3). FIG. 2 shows the continuous evolution from day 1 to day 28 of the emulsion layer without HA and the aqueous phase below the emulsion layer. The rapid formation of creaming is observed, as well as a lightening of the aqueous phase with time. In FIG. 3, the emulsion layer occupies almost the entire sample and remains stable for 28 days due to the presence of HA in the outer aqueous phase. HA acts as a thickener and stabilizer of the outer aqueous phase.

The viscosity of emulsions with and without HA in the external phase was studied and compared to the viscosity of an emulsion containing Carbopol at 0.2% by mass as a thickening agent in the aqueous external phase. The emulsion containing Carbopol has a suitable texture for the desired applications, and its viscosity is therefore used as a reference.

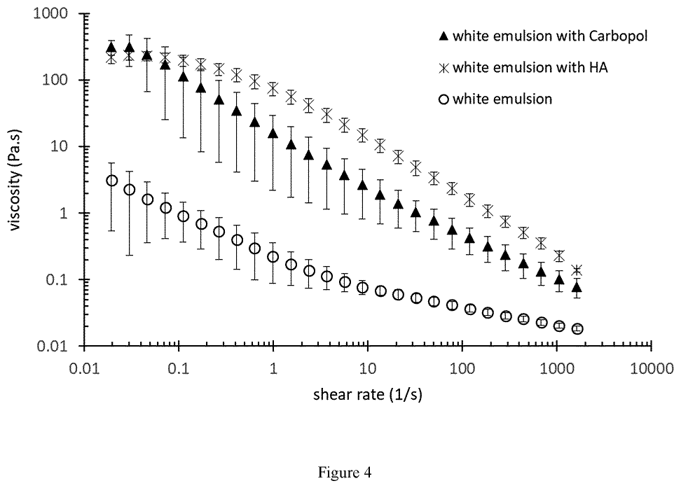

FIG. 4 shows the evolution of the viscosity of a white emulsion (i.e. without any active substance) as a function of the shear rate, containing HA in the external phase. This curve is compared with that of a white emulsion without thickener in the external phase, and with that of an emulsion containing 0.2% Carbopol in the external phase.

We can observe a shear-thinning behavior of all emulsions (viscosity decreases when the shear rate increases). The addition of a thickener, AH or Carbopol, significantly increases the viscosity of the emulsion over the whole range of shear rates studied. The viscosity of the emulsion containing 1.5% HA in the outer phase is comparable to the viscosity of the emulsion containing 0.2% Carbopol (or even slightly higher for shear rates above 0.2 s-1).

It was also possible to obtain stable emulsions with other concentrations of hyaluronan, including with concentrations such as 0.5%, 1%, or 1.25% w/vol in hyaluronan in the aqueous suspension (i.e. 0.5, 1, or 1.25 g of HA per 100 ml of the aqueous phase). This allows to provide stable emulsions displaying various viscosity profiles.

### In Vivo Effects of Carbopol Emulsions on Murine Imiquimod (IMQ)-Induced Psoriasis Model

After specific induction of psoriasis-like dermatose by IMQ in BALB/c mice, biological effects and “cutaneous” scores of emulsions (white control vs AS emulsions) were studied for 7 days

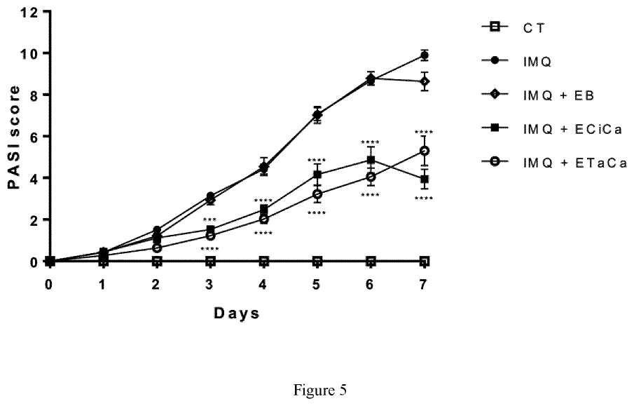

Results are presented on FIG. 5 as score PASI means±SEM from groups of twelve mice each, where the combined whole emulsion including Calcitriol (Ca) and the active immunosuppressive drug (ciclosporin A or tacrolimus), namely (ECiCa) and (ETaCa) are compared to the “white emulsion” (EB for Emulsion “Blanche”) without active substance.

### In Vivo Effects of HA Emulsions on Murine Imiquimod (IMQ)-Induced Psoriasis Model

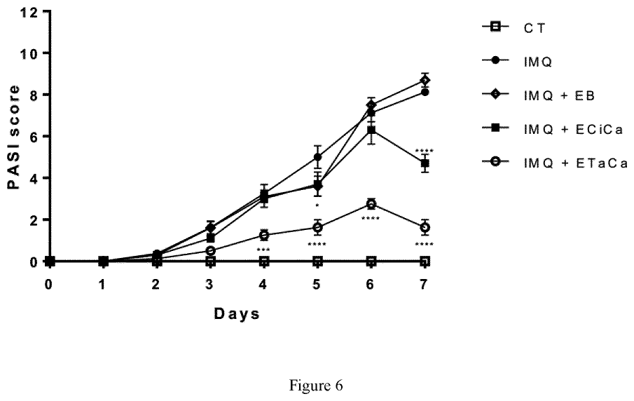

The influence of the addition of hyaluronic acid (HA) with a molar mass of 1.5 MDa in the external phase of the emulsion was compared to carbopol emulsions in the mouse model of psoriasis induced by imiquimod in vivo (FIG. 6).

Calculated as PASI Score in mice phenotypically resembles psoriasis. BALB/c mice were treated daily with IMQ cream or control cream on the shaved back skin and right ear. Erythema, scaling, and thickness of the back skin was scored daily on a scale from 0 to 4. Results are expressed as the cumulative score (erythema plus scaling plus thickness) depicted and reported here as the Score PASI total (ordinate).

Our present results clearly demonstrate the clinical efficiency of the innovative Pickering emulsions in IMQ-induced psoriasiform dermatosis.

No significant differences were detected in vivo on the IMQ-induced psoriasis in the presence of the “carbopol-prepared emulsions”compared to the “HA prepared emulsions”.

In the case of the ETaCa emulsion “HA prepared emulsions” seem to have a better effect than “carbopol-prepared emulsions”.

1. Oil-in-water Pickering emulsion comprising:
an oil phase comprising a first therapeutic agent,
an aqueous phase,
polyester nanoparticles comprising a second therapeutic agent,
wherein the oil phase is in the form of droplets and is dispersed in a continuous aqueous phase, and wherein at least a portion of the nanoparticles are localized at an interface between the oil phase and the aqueous phase,
and wherein the aqueous phase comprises hyaluronan.

2. The oil-in-water Pickering emulsion according to claim 1, wherein the pH of the emulsion is from 4.5 to 6.5.

3. The oil-in-water Pickering emulsion according to claim 1, wherein the oil phase comprises at least one oil chosen from fatty acids, fatty acid esters, vegetable oils, mineral oils, and mixtures thereof.

4. The oil-in-water Pickering emulsion according to claim 3, wherein the fatty acid esters are selected from triglycerides of medium-chain fatty acids.

5. The oil-in-water Pickering emulsion according to claim 1, wherein the droplets forming the oil phase have a size distribution ranging from 1 μm to 100 μm, as measured by dynamic light scattering.

6. The oil-in-water Pickering emulsion according to claim 1, wherein the polyester is selected from polylactic acid, polyglycolic acid, copolymers of lactic acid and glycolic acid, copolymers of lactic acid, glycolic acid and ethylene glycol, polyorthoesters, polyanhydrides, polylactones, polymalic acid, and mixtures thereof.

7. The oil-in-water Pickering emulsion according to claim 6, wherein the polyester is poly(lactic-co-glycolic acid).

8. The oil-in-water Pickering emulsion according to claim 1, wherein the first therapeutic agent is selected from anti-inflammatory agents and natural or semisynthetic analogs derived from metabolites of vitamin E.

9. The oil-in-water Pickering emulsion according to claim 1, wherein the second therapeutic agent is an immunosuppressive agent.

10. The oil-in-water Pickering emulsion according to claim 1, wherein the oil phase further comprises an antioxidant.

11. The oil-in-water Pickering emulsion according to claim 1, wherein the molar mass of hyaluronan is from 5000 g/mol to 5000000 g/mol.

12. The oil-in-water Pickering emulsion according to claim 1, wherein the concentration of hyaluronan in the aqueous phase calculated as the mass of hyaluronan in grams contained in 100 mL of the aqueous phase is from 0.15% to 5%.

13. The oil-in-water Pickering emulsion according to claim 1, wherein
the first therapeutic agent is Tacrolimus, and
the second therapeutic agent is calcitriol.

14. Pharmaceutical composition comprising the oil-in-water Pickering emulsion according to claim 1 and at least one pharmaceutically acceptable excipient.

15. A method of treating a dermatologic pathology in a human or animal in need thereof, comprising administering to the human or animal a therapeutically effective amount of the oil-in-water Pickering emulsion of claim 1.
